(19) United States
(12) Patent Application Publication
(10) Pub. No.: US 2009/0254103 A1
(43) Pub. Date: Oct. 8, 2009

(54) METHOD AND DEVICE FOR CAVITY OBLITERATION

(76) Inventor: Harvey L. Deutsch, Los Angeles, CA (US)

Correspondence Address: SHELDON MAK ROSE & ANDERSON PC, 100 Corson Street, Third Floor, PASADENA, CA, 91103-3842, US

(21) Appl. No.: 11/577,987

(22) PCT Filed: Mar. 29, 2007
(86) PCT No.: PCT/US07/65527

§ 371 Date: Apr. 25, 2007

Related U.S. Application Data
(60) Provisional application No. 60/743,922, filed on Mar. 29, 2006.
(60) Provisional application No. 60/744,549, filed on Apr. 10, 2006.
(60) Provisional application No. 60/745,349, filed on Apr. 21, 2006.

(51) Int. Cl. A61B 17/03

(52) U.S. Cl. 606/151

(57) ABSTRACT

A device for the obliteration of an aberrant space or cavity. A method for the obliteration of an aberrant space or cavity comprising providing a device according to the present invention.

### CROSS-REFERENCE TO RELATED APPLICATIONS

The present application claims the benefit of U.S. Provisional Patent Application No. 60/743,922 titled “Method and Device for Cavity Obliteration” filed Mar. 29, 2006; U.S. Provisional Patent Application No. 60/744,549 titled “Method and Device for Cavity Obliteration” filed Apr. 10, 2006; and U.S. Provisional Patent Application No. 60/745,349 titled “Method and Device for Cavity Obliteration” filed Apr. 21, 2006, the contents of which are incorporated in this disclosure by reference in their entirety.

### BACKGROUND

There are a number of human diseases and conditions that include the existence of an aberrant space or cavity, such as, for example, a hernia sac of an inguinal hernia, and for which treatment of the disease or condition involves obliteration of the space or cavity. Generally, obliteration of the hernia sac of an inguinal hernia is accomplished by an open procedure where the connection between the sac and the peritoneal cavity is visualized, and the sac is then obliterated under direct visualization. There are, however, a number of disadvantages to open surgical approaches for the repair of inguinal hernias, including the need for a moderately sized skin incision that involves a significant risk of wound dehiscence, infection, post-operative pain, hernia recurrence, and an extended recuperative period.

Therefore, there remains a need for a new method for the obliteration of an aberrant space or cavity, such as the hernia sac of an inguinal hernia.

### SUMMARY

According to one embodiment of the present invention, there is provided a device for the obliteration of an aberrant space or cavity. The device comprises: a) a patch portion comprising a first side and an opposing second side; b) a plurality of struts arranged radially between the first side and the second side, where each strut comprises a first end and a second end; and c) a clip associated with the second end of each strut; where the patch portion has a center and a circumference; where the first end of each strut is oriented toward the center of the patch portion, and the second end is oriented toward the circumference of the patch portion; where each clip is configured to attach the patch portion to a surface or structure adjacent to or within the aberrant space or cavity to be obliterated; and where each strut comprises two lumens, a first lumen configured to contain a stabilization wire, and a second lumen configured to contain an attachment line. In one embodiment, the device comprises between 2 and 20 struts. In another embodiment, the device comprises between 5 and 8 struts. In one embodiment, the patch portion is convex toward the first side and comprises a generally circular circumference. In one embodiment, the second side comprises visually discernable markings corresponding to the position of each clip. In one embodiment, each strut is curved or bent to create the convex shape of the patch portion. In one embodiment, each clip comprises a plurality of arms comprising a first end and a second end, where the first ends of each arm of each clip are joined together, and are also connected to an attachment line. In one embodiment, the second end of each arm comprises one or more than one gripping tip. In one embodiment, each attachment line is joined at the proximal end to form a collective attachment line allowing simultaneous closing of the second ends of the arms of multiple clips.

According to another embodiment of the present invention, there is provided a deployment system for deploying a device according to the present invention. In one embodiment, the device comprises: a) a device according to the present invention; b) a deployment catheter comprising a proximal end and a distal end, and configured to advance over a guidewire; and c) one stabilization wire within each of the plurality of struts, where the stabilization wires are joined to a central pull wire within the deployment catheter.

According to another embodiment of the present invention, there is provided a device for the obliteration of an aberrant space or cavity, the device comprising: a) a patch portion comprising a first side and an opposing second side; and b) a frame portion comprising a first side and an opposing second side; where the first side of the patch portion comprises a first surface of two opposing surfaces of a hook and loop fastener; where the second side of the frame portion further comprises a second surface of two opposing surfaces of a hook and loop fastener configured to mate with the first surface of the first side of the patch portion; where the frame portion further comprises a plurality of peripherally radiating members, each of the peripherally radiating members comprising a first end and a second end; where the first ends of each radiating member are joined together; and where each of the second ends of the peripherally radiating members comprises a structure to attach the radiating member, and hence the frame portion, to a surface or structure adjacent to or within the aberrant space or cavity to be obliterated.

According to another embodiment of the present invention, there is provided a device for the obliteration of an aberrant space or cavity, the device comprising: a) a patch portion comprising a first side, an opposing second side, and an outer edge; b) an inflation area or inflation channel, which when inflated, gives shape to the patch portion in a post-deployment configuration; c) an adhesive delivery channel comprising a series of perforations to allow adhesive to exit from the adhesive delivery channel; and d) one or more than one valve for introducing inflation material into the inflation area or inflation channel and for introducing adhesive into the adhesive delivery channel. In one embodiment, the inflation area or inflation channel is a circumferential conduit arrayed just central to the outer edge. In another embodiment, the inflation area or inflation channel comprises a single, centrally orientated inflation arm. In another embodiment, the inflation area or inflation channel comprises a single, centrally orientated inflation arm, and the adhesive delivery channel comprises a single, centrally orientated adhesive delivery arm, and the one or more than one valve is positioned in the center of the patch portion in continuity with the centrally orientated inflation arm and the centrally orientated adhesive delivery arm. In one embodiment, the patch portion further comprises a peripheral flange, oriented peripherally to the inflation area or inflation channel to allow the device to be fixed into position through the flange. In one embodiment, the device further comprises a suture harness attached to the first side of the patch portion. In one embodiment, the patch portion further comprises a peripheral flange, oriented peripherally to the inflation area or inflation channel to allow the device to be fixed into position through the flange; where the device further comprises a suture harness attached to the first side of the patch portion.

In one embodiment, the first side, the second side or both the first side and the second side of the patch portion comprise material selected from the group consisting of polypropylene, polypropylene mesh, polytetrafluoroethylene (PTFE) graft material and silicone rubber.

According to another embodiment of the present invention, there is provided a method for the obliteration of an aberrant space or cavity comprising an open end and a closed end. The method comprises: a) selecting an aberrant space or cavity that is suitable for obliteration by the method; b) providing a device according to the present invention; and c) deploying the device to substantially seal the open end of the aberrant space or cavity. In one embodiment, the aberrant space or cavity obliterated by the method is within a living organism. In another embodiment, the aberrant space or cavity obliterated by the method is within a human. In another embodiment, the aberrant space or cavity obliterated by the method is a hernia sac of an inguinal hernia. In one embodiment, the method further comprises creating an opening in the closed end of the aberrant space or cavity and introducing the device through the opening in the closed end of the aberrant space or cavity. In one embodiment, the aberrant space or cavity is the hernia sac of an inguinal hernia, where the hernia sac is covered by skin, and where deploying the device comprises: a) inducing anesthesia; b) distending the hernia sac with carbon dioxide gas; and c) inserting a trocar into the intraperitoneal cavity adjacent the inguinal hernia. In another embodiment, the method further comprises: a) providing an adaptor sheath having two proximal self-sealing valves; b) introducing the sheath through a trocar; c) introducing a laparoscope through the sheath; d) introducing a wire snare into the intraperitoneal cavity through the sheath; e) providing a guidewire having a proximal end and a distal end, and inserting the proximal end of the guidewire through the skin over the hernia sac; f) advancing the proximal end of the guidewire into the intraperitoneal cavity; g) capturing the proximal end of the guidewire with the wire snare; h) pulling the proximal end of the guidewire through the trocar; i) providing a delivery device comprising a capsule having a proximal end and a distal end and a pusher, and containing a deployment system comprising the device in a pre-deployment configuration within the capsule; j) advancing the delivery device containing the deployment system over the guidewire through the trocar using the delivery catheter in monorail fashion until the distal end of the capsule contacts the area surrounding the hernia sac; k) releasing the deployment system with the device from the delivery device by maintaining the pusher in place and retracting the capsule proximally; l) changing the patch portion of the device to a post-deployment configuration; m) withdrawing the delivery device from the trocar; and n) positioning the patch portion of the device, thereby effecting the obliteration of the aberrant space or cavity.

According to another embodiment of the present invention, there is provided a method for the obliteration of an aberrant space or cavity comprising an open end and a closed end, the method comprising: a) selecting an aberrant space or cavity that is suitable for obliteration by the method; b) providing a device according to the present invention; c) deploying the device to substantially seal the open end of the aberrant space or cavity; d) inflating the inflation area or inflation channel of the patch portion of the device to impart structural rigidity to the patch portion; and e) introducing adhesive through the adhesive delivery conduit and allowing the adhesive to discharge from the series of perforations in the adhesive delivery channel.

### FIGURES

These and other features, aspects and advantages of the present invention will become better understood with regard to the following description, appended claims, and accompanying figures which depict various views and embodiments of the device, and some of the steps in certain embodiments of the method of the present invention, where:

FIG. 1 is a lateral perspective view of one embodiment of the device for the obliteration of an aberrant space or cavity in a pre-deployment configuration;

FIG. 2 is a lateral perspective view of the embodiment of the device shown in FIG. 1 in a post-deployment configuration;

FIG. 3 is a lateral perspective view of the frame portion of the device shown in FIG. 2 in a post-deployment configuration;

FIG. 4, FIG. 5 and FIG. 6 are close-up, lateral perspective views of one embodiment of a clip suitable for incorporation into the embodiment shown in FIG. 1, FIG. 2 and FIG. 3;

FIG. 7 is a partial cross-sectional view of an aberrant space or cavity after having been sealed by the device as shown in FIG. 1 and FIG. 2;

FIG. 8 is a top perspective view of the first side of the patch portion of another embodiment of the device for the obliteration of an aberrant space or cavity in a post-deployment configuration;

FIG. 9 is a bottom perspective view of the second side of the patch portion of the embodiment of the device shown in FIG. 8 in a post-deployment configuration;

FIG. 10 is a lateral perspective view of the patch portion of the device shown in FIG. 8 and FIG. 9 in a post-deployment configuration;

FIG. 11 is a lateral perspective view of a strut and clip of the device shown in FIG. 9;

FIG. 12 is a cross-sectional view of the strut shown in FIG. 11;

FIG. 13 is a close-up, partial, cutaway, perspective view of the second end of the strut shown in FIG. 11 shown in the pre-deployment configuration;

FIG. 14 is a close-up, partial, cutaway, perspective view of the second end of the strut shown in FIG. 11 shown in the post-deployment configuration;

FIG. 15 is a lateral perspective view of a stabilization wire used in the device;

FIG. 16 is a lateral perspective view of the strut and clip of the device shown in FIG. 11 with the stabilization wire shown in FIG. 15 in position within the strut;

FIG. 17 is a cross-sectional view of the mid section of the strut shown in FIG. 16;

FIG. 18 is a lateral perspective view of a group of stabilization wires, as shown in FIG. 15, joined together centrally by a fitting to a central pull wire;

FIG. 19 is a partial, lateral perspective view of a deployment system according to the present invention;

FIG. 20, FIG. 21 and FIG. 22 are partial, lateral perspective views of the stabilization wires, central pull wire and metal fitting as they are retracted into the proximal end of the deployment catheter;

FIG. 23, FIG. 24 and FIG. 25 are partial lateral perspective views of the proximal end of the deployment system, showing the device of the present invention being rotated for proper placement, the stabilization wires being withdrawn from the struts into the proximal end of the deployment catheter, and the deployment catheter with the struts inside being separated from the device;

FIG. 26 is a cross-sectional view of an aberrant space or cavity suitable for obliteration by a method for the obliteration of an aberrant space or cavity according to the present invention;

FIG. 27, FIG. 30, FIG. 31 and FIG. 33 through FIG. 37 are cross-sectional views of various steps in a method for the obliteration of an aberrant space or cavity according to the present invention;

FIG. 28 is a lateral perspective view of an adaptor sheath for use in the present method;

FIG. 29 is a lateral perspective view of a wire snare for use in the present method;

FIG. 32 is a partial, lateral perspective view of a delivery device suitable for use in the present method;

FIG. 38 is a lateral perspective view of the second side of the patch portion of another embodiment of the device for the obliteration of an aberrant space or cavity in a post-deployment configuration;

FIG. 39 is a top perspective view of the frame portion of the embodiment of the device shown in FIG. 38 in a post-deployment configuration;

FIG. 40 is a bottom perspective view of the patch portion of the device shown in FIG. 38 and the frame shown in FIG. 39 in a post-deployment configuration, where the patch portion and frame portion are joined together;

FIG. 41 is a partial lateral perspective view of the opposing surfaces of a hook and loop fastener with the two sides in the unattached configuration;

FIG. 42 is a partial lateral perspective view of the opposing surfaces of a hook and loop fastener as shown in FIG. 41 with the two sides in the attached configuration;

FIG. 43 is a top perspective view of the first side of the patch portion of another embodiment of the device for the obliteration of an aberrant space or cavity in a post-deployment configuration;

FIG. 44 is a lateral perspective view of the patch portion of the device shown in FIG. 43 in a post-deployment configuration;

FIG. 45 is a lateral perspective view of the patch portion and optional suture harness of the device shown in FIG. 43 in a post-deployment configuration;

FIG. 46 is a cross-sectional view of one step in the present method for the obliteration of an aberrant space or cavity according to the present invention;

FIG. 47 is a partial, close-up lateral perspective view of the distal end of the deployment catheter as shown in FIG. 46;

FIG. 48 is a partial, close-up lateral perspective view of the distal end of the deployment catheter as shown in FIG. 47 with the distal portion of the deployment catheter removed;

FIG. 49 is a partial, close-up lateral perspective view of the distal end of the deployment catheter as shown in FIG. 48 with luer lock hubs attached to the inflation material delivery conduit and to the adhesive delivery conduit;

FIG. 50 and FIG. 51 are sequential, partial close-up lateral perspective views of the patch portion of the device in the post-deployment configuration being separated from the proximal portion of the deployment catheter, the inflation material delivery conduit, the adhesive delivery conduit and the pusher rod according to the embodiment of the method shown in FIG. 46.

### DESCRIPTION

According to one embodiment of the present invention, there is provided a device for the obliteration of an aberrant space or cavity. In a preferred embodiment, the aberrant space or cavity is within a living organism, such as within a human. In another preferred embodiment, the aberrant space or cavity is a hernia sac of an inguinal hernia. In another preferred embodiment, the aberrant space or cavity is a vascular or cardiac aneurysm. According to another embodiment of the present invention, there is provided a method for the obliteration of an aberrant space or cavity comprising an open end and a closed end. In a preferred embodiment, the aberrant space or cavity obliterated by the method is within a living organism, such as within a human. In another preferred embodiment, the aberrant space or cavity obliterated by the method is a hernia sac of an inguinal hernia. In another preferred embodiment, the aberrant space or cavity is a vascular or cardiac aneurysm. In one embodiment, the method comprises providing a device according to the present invention. In another embodiment, the method comprises deploying a device through the opening created in the closed end of the aberrant space or cavity.

As used in this disclosure, the term “comprise” and variations of the term, such as “comprising” and “comprises,” are not intended to exclude other additives, components, integers or steps.

As used in this disclosure, the term “closed end of the aberrant space or cavity” means any position on the wall of the aberrant space or cavity other than through the open end of the aberrant space or cavity.

As used in this disclosure, the term “obliterate” means to substantially seal the open end of the aberrant space or cavity.

All dimensions specified in this disclosure are by way of example only and are not intended to be limiting. Further, the proportions shown in these Figures are not necessarily to scale. As will be understood by those with skill in the art with reference to this disclosure, the actual dimensions of any device or part of a device disclosed in this disclosure will be determined by its intended use.

The method steps disclosed in this disclosure are not intended to be limiting nor are they intended to indicate that each step depicted is essential to the method, but instead are exemplary steps only.

According to one embodiment of the present invention, there is provided a device for the obliteration of an aberrant space or cavity. In a preferred embodiment, the aberrant space or cavity is within a living organism, such as within a human. In another preferred embodiment, the aberrant space or cavity is a hernia sac of an inguinal hernia. In another preferred embodiment, the aberrant space or cavity is a vascular or cardiac aneurysm.

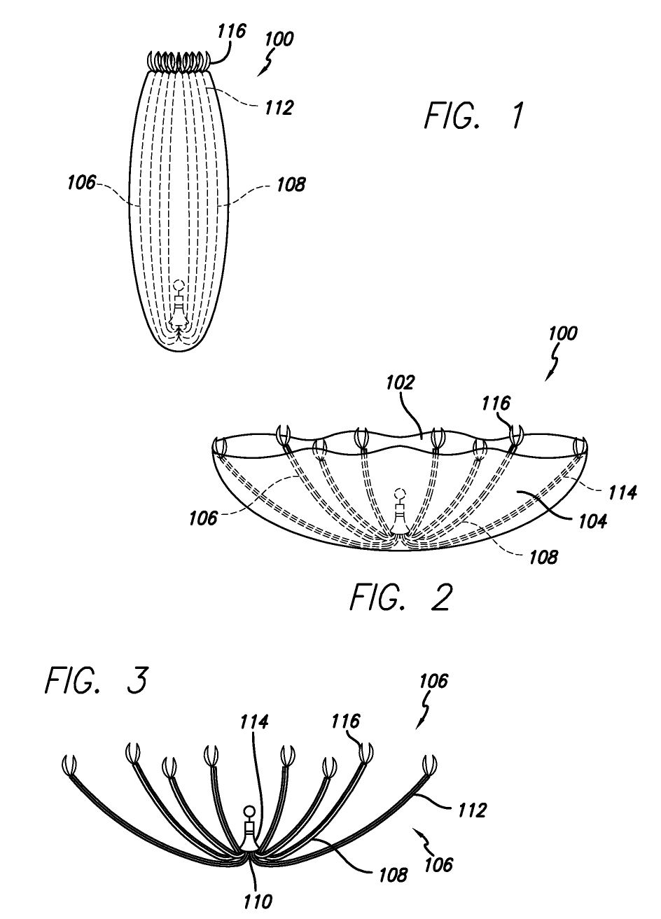

Referring now to FIG. 1, FIG. 2 and FIG. 3, there are shown, respectively, a lateral perspective view of one embodiment of the device for the obliteration of an aberrant space or cavity in a pre-deployment configuration (FIG. 1); a lateral perspective view of the embodiment of the device shown in FIG. 1 in a post-deployment configuration (FIG. 2); and a lateral perspective view of the frame portion of the device shown in FIG. 2 in a post-deployment configuration (FIG. 3). As can be seen, in this embodiment, the device 100 comprises a first side 102, an opposing second side 104, and a frame 106 between the first side 102 and the second side 104.

In one embodiment, the first side 102 and the second side 104 comprise material selected from the group consisting of polypropylene, polytetrafluoroethylene (PTFE) graft material and silicone rubber. In a preferred embodiment, such as when the device 100 is being used to obliterate an inguinal hernia sac in a human, the first side 102 comprises polypropylene and the second side 104 comprises polytetrafluoroethylene. The first side 102 and the second side 104 can, however, comprise any suitable material, as will be understood by those with skill in the art with reference to this disclosure.

The frame 106 comprises a plurality of peripherally radiating members 108 comprising a first end 110 and a second end 112. The first end 110 of each radiating member 108 is joined at a central connector 114. The second end 112 of one or more than one of the radiating members 108 comprises a clip 116 to attach the radiating member 108 to a surface or structure adjacent to or within the aberrant space or cavity to be obliterated, such as to the peritoneal surface at the entry site into the hernia sac of an inguinal hernia, thereby immobilizing the device 100 in position.

As can be seen with particular reference to FIG. 1, when this embodiment is in a pre-deployment configuration, the second ends 112 of the radiating members 108 approximate, thereby rendering the device 100 into a smaller axial profile than in a post-deployment configuration suitable for deployment through a small opening. As can be seen with particular reference to FIG. 2, in a preferred embodiment, when the device is deployed, the first side 102 and the second side 104 form a patch that is concave toward the first side 102 and that comprises a generally circular circumference, though other configurations are suitable for various uses of the device 100, as will be understood by those with skill in the art with reference to this disclosure.

As can be seen with particular reference to FIG. 2 and FIG. 3, after deployment, the second ends 112 of the radiating members 108 are separated by actuating a mechanism in the central connector 114, thereby rendering the device 100 into a post-deployment configuration suitable for sealing the opening of the aberrant space or cavity. In one embodiment, the frame 106 comprises wire, such as a shaped metal alloy or is a shaped memory polymer, such as a suitable polystyrene material. In a preferred embodiment, the metal alloy is selected from the group consisting of a nitinol and a cobalt-chromium-nickel-molybdenum-iron alloy specified by ASTM F1058 and ISO 5832-7, stainless steel or a ferrous alloy containing cobalt, chromium, nickel, molybdenum, manganese, carbon, and beryllium, such as Elgiloy® (RMO, Denver, Colo. US). In a preferred embodiment, such as when the device 100 is used to obliterate an aberrant space or cavity within a human, the frame 106 comprises wire of a biocompatible material.

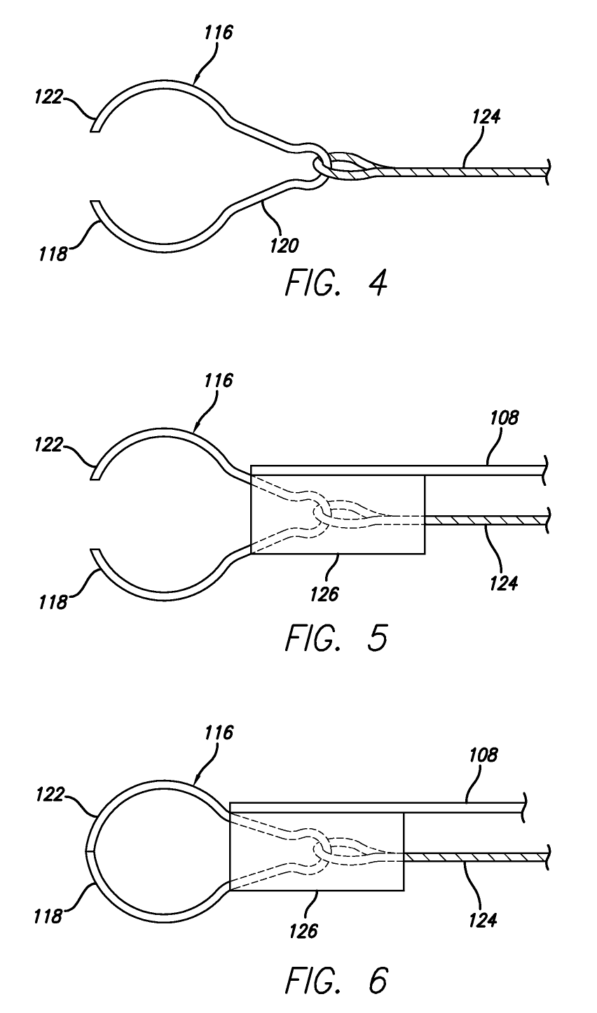

Referring now to FIG. 4, FIG. 5 and FIG. 6, there are shown, respectively, close-up, lateral perspective views of parts of one embodiment of a clip 116 suitable for incorporation into the embodiment shown in FIG. 1, FIG. 2 and FIG. 3. As will be understood by those with skill in the art with reference to this disclosure, other clips are also suitable for incorporation into the device 100. In the embodiment shown in FIG. 4, FIG. 5 and FIG. 6, the clip 116 comprises a plurality of arms 118 comprising a first end 120 and a second end 122. The first ends 120 of each arm 118 of each clip 116 are joined together, and are also joined to an attachment line 124. In a preferred embodiment, the second end 122 of each arm 118 comprises one or more than one gripping tip, such as, for example, a sharp point or barb. Though shown with only two arms 118, each clip 116 can comprise three or more arms, as will be understood by those with skill in the art with reference to this disclosure. Each clip 116 further comprises a tubular structure 126 surrounding the first end 120 and attached to the frame 106. In one embodiment, each clip 116 comprises tantalum, titanium or another suitable metal; however, each clip 116 can comprise any suitable material, as will be understood by those with skill in the art with reference to this disclosure.

As can be seen with particular reference to FIG. 5, in the pre-deployment position, the second ends 122 of each arm 118 are separated from each other and extend maximally outside the tubular structure 126. As can be seen with particular reference to FIG. 6, after deployment, axial force is applied to the attachment line 124, toward the central connector 114 of the frame 106, thereby translating the joined first ends 120 of the clip 116 axially toward the central connector 114, and approximating the second ends 122 of the clip 116. Any surface or structure adjacent to or within the aberrant space or cavity to be obliterated between the second ends 122 of the clip 116 at the time of deployment is then caught between the second ends 122, thereby immobilizing the device 100 in position. In a preferred embodiment, the second side 104 comprises visually discernable markings corresponding to the placement of each clip 116.

According to another embodiment of the present invention, there is provided a method for the obliteration of an aberrant space or cavity comprising an open end and a closed end. In a preferred embodiment, the aberrant space or cavity obliterated by the method is within a living organism, such as within a human. In another preferred embodiment, the aberrant space or cavity obliterated by the method is a hernia sac of an inguinal hernia. In one embodiment, the method comprises providing a device according to the present invention, and deploying the device to substantially seal the open end of the aberrant space or cavity. In another embodiment, the method comprises creating an opening in the closed end of the aberrant space or cavity, introducing a device through the opening in the closed end of the aberrant space or cavity, and deploying the device to substantially seal the open end of the aberrant space or cavity. In a preferred embodiment, the device introduced is a device according to the present invention.

By way of example, the method will now be disclosed in greater detail with specific reference to the obliteration of a hernia sac of an inguinal hernia. As will be understood by those with skill in the art with reference to this disclosure, however, equivalent steps can be used to obliterate any aberrant space or cavity suitable for obliteration by the present method, including an aberrant space or cavity other than the hernia sac of an inguinal hernia, other than within a living organism, and other than within a human.

As will be appreciated by one with skill in the art with reference to this disclosure, when the method of the present invention is used to obliterate the hernia sac of an inguinal hernia, the method preferably involves a percutaneous transcatheter approach, though a laparoscopic approach or open surgical approach can also be used. Specifically, in a preferred embodiment, the method comprises deploying a device through an opening created in the closed end of the aberrant space or cavity using a percutaneous transcatheter approach. Compared with open surgical and laparoscopic approaches for the treatment of inguinal hernias currently performed, the percutaneous transcatheter approach of the present method reduces procedure times, decreases risks of infection, requires smaller incisions and fewer punctures, and reduces recuperation time. Further, the method reduces procedural costs due to the utilization of an interventional radiology suite for the repair instead of a more expensive operating room environment.

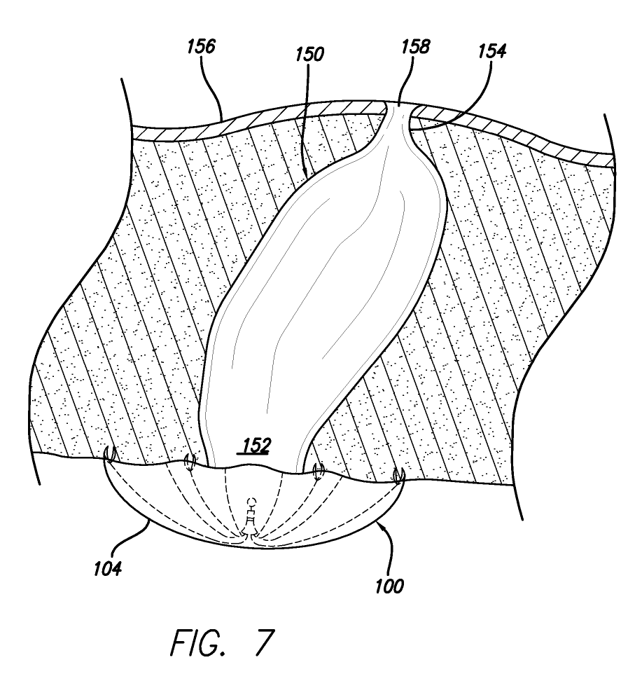

Referring now to FIG. 7, there is shown a partial cross-sectional view of an aberrant space or cavity after having been obliterated by the device according to the present invention as shown in FIG. 1 and FIG. 2. The steps disclosed are not intended to be limiting nor are they intended to indicate that each step depicted is essential to the method, but instead are exemplary steps only.

The method comprises, first selecting an aberrant space or cavity 150 that is suitable for obliteration by the method. The aberrant space or cavity 150 comprises an open end 152 and a closed end 154. In one embodiment, the aberrant space or cavity 150 is within a living organism. In a preferred embodiment, the aberrant space or cavity 150 is within a human. In a particularly preferred embodiment, the aberrant space or cavity 150 is a hernia sac of an inguinal hernia within a human.

In one embodiment, selecting an aberrant space or cavity 150 that is suitable for obliteration by the method comprises selecting a patient having a disease or condition that includes the existence of an aberrant space or cavity 150, such as, for example, the hernia sac of an inguinal hernia, and for which treatment of the disease or condition involves obliteration of the aberrant space or cavity 150. In this embodiment, selecting the patient comprises diagnosing the existence of an aberrant space or cavity 150 using standard techniques, such as a technique selected from the group consisting of CT scan, herniography, history, MRI and physical examination.

The following steps are disclosed with respect to obliterating the hernia sac 150 of an inguinal hernia as an example. Next, anesthesia is induced, and the lower abdomen and inguinal areas prepped and draped in a sterile fashion, according to standard techniques. Then, an opening 158 is created in the closed end 154 of the hernia sac 150. A device for obliterating the hernia sac 150 is deployed, thereby obliterating the hernia sac 150. In a preferred embodiment, the device deployed is a device 100 for the obliteration of an aberrant space or cavity 150 according to the present invention. In another preferred embodiment, the device is introduced percutaneously. Introduction of the device can be accomplished in a number of ways depending on the embodiment of the device used, as will be understood by those with skill in the art with reference to this disclosure.

By way of example only, various introduction steps will now be disclosed in detail. A puncture incision is made in the skin of the lower abdomen 156 with a 20-22 gauge needle. In one embodiment, the peritoneal cavity is inflated with a suitable gas, such as, for example, carbon dioxide gas, which also distends the hernia sac 150. Then, the closed end 154 of the distended hernia sac 150 is then entered by a second incision, such as a puncture incision, with an 18-gauge needle creating an opening 158 in the closed end 154. A 1 mm diameter guidewire is advanced under suitable guidance, such as, for example, fluoroscopic guidance, through the opening 158 in the closed end 154 of the hernia sac 150, through the hernia sac 150, and through the open end 152 of the hernia sac 150 into the peritoneal cavity. Next, the needle is removed, and over the guidewire, a 12 F to 14 F (4.0 mm to 4.7 mm) introducer catheter with its central dilator is advanced through the opening 158 in the closed end 154 of the hernia sac 150, through the hernia sac 150, and through the open end 152 of the hernia sac 150 into the peritoneal cavity. The guidewire and central dilator are then removed.

Then, a device 100 according to the present invention in its pre-deployment configuration is advanced into the hernia sac 150. In a preferred embodiment, the device 100 is advanced through the introducer catheter directly. Introducing the device 100 into the hernia sac 150, whether through the introducer catheter or not, can comprise collapsing the device 100 by bringing the perimeter of the device 100 toward the center, or by another method as will be understood by those with skill in the art with reference to this disclosure. The device 100 is deployed at or near the open end 152 of the hernia sac 150 at the junction of the peritoneal cavity. As appropriate for the embodiment of the device 100, the device 100 can be attached to a pusher rod to assist in proper placement of the device 100 during deployment. Referring again to FIG. 7, when deployed, the clips 116 attach the frame 106, and hence the device 100, to the peritoneal surface adjacent to or within the hernia sac 150, thereby immobilizing the device 100 and obliterating the cavity 150.

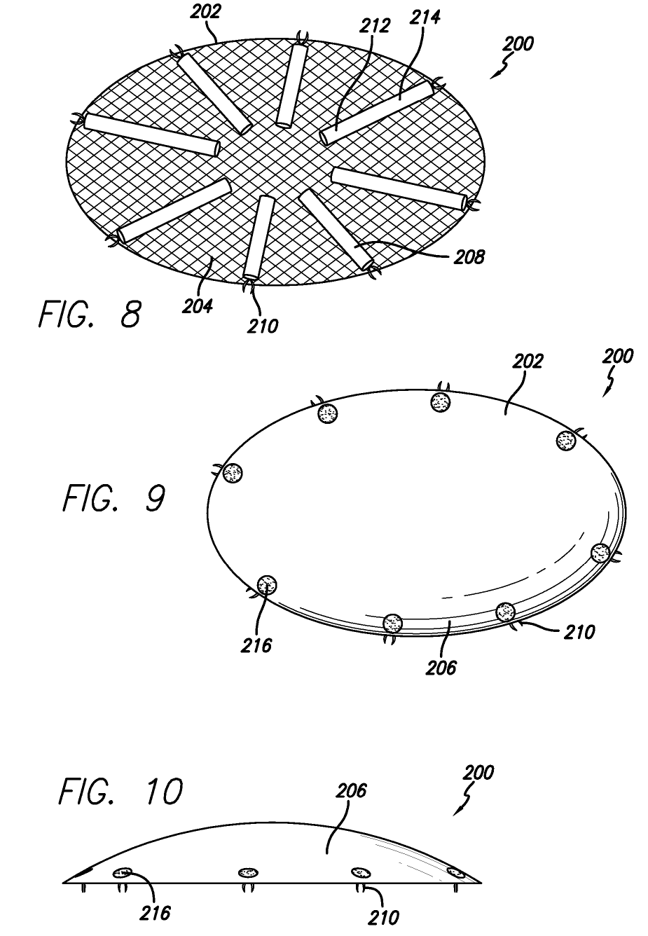

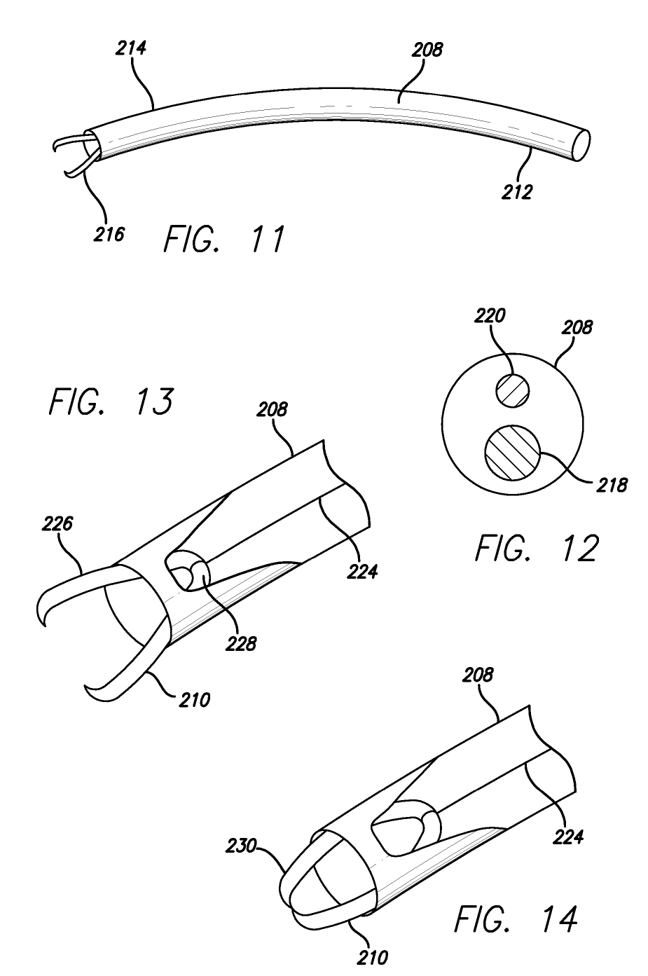

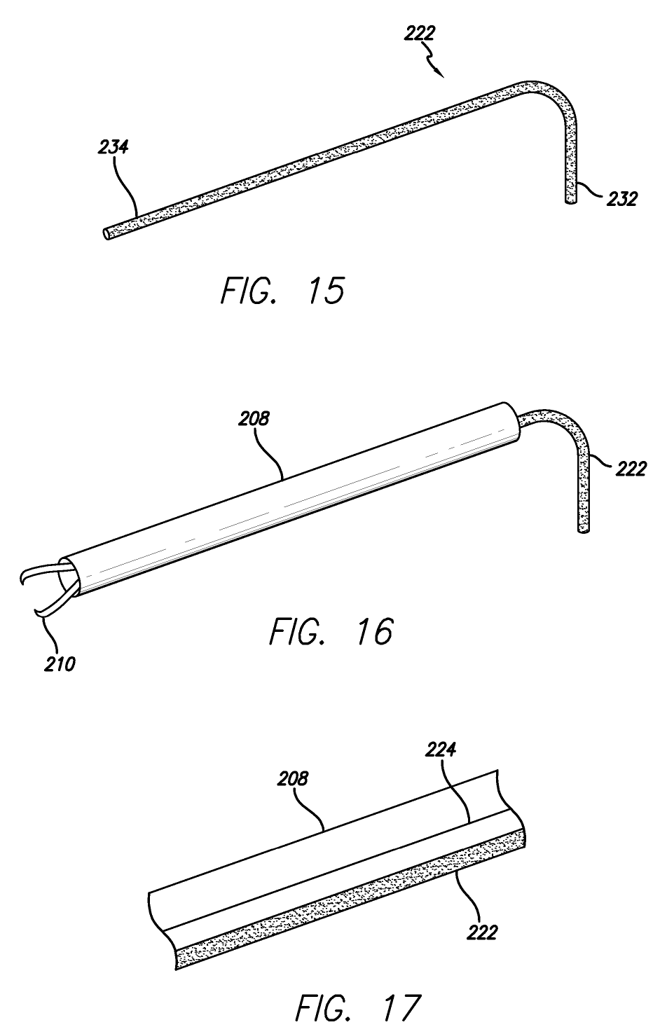

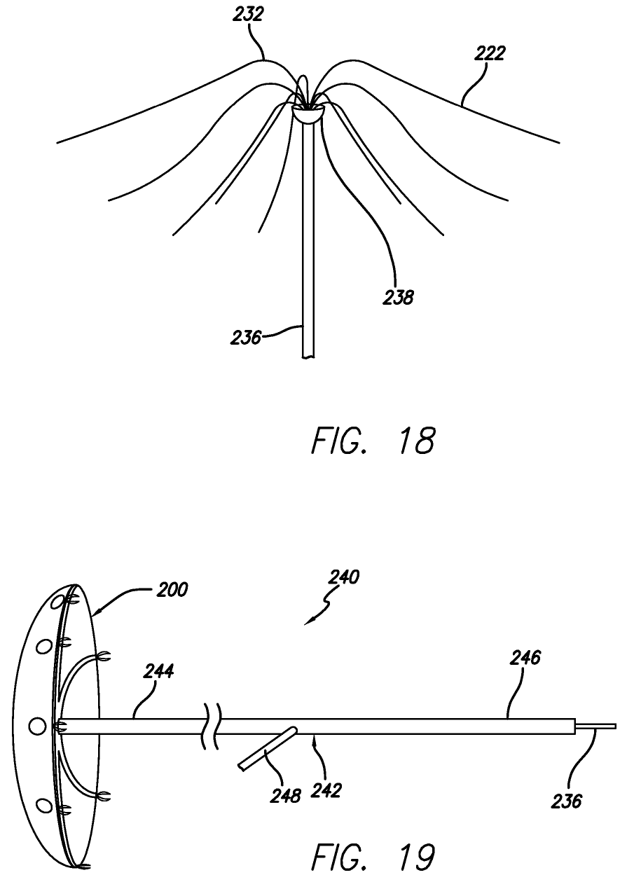

Referring now to FIG. 8 through FIG. 18, there are shown, respectively, a top perspective view of the first side of the patch portion of another embodiment of the device for the obliteration of an aberrant space or cavity in a post-deployment configuration (FIG. 8); a bottom perspective view of the second side of the patch portion of the embodiment of the device shown in FIG. 8 in a post-deployment configuration (FIG. 9); a lateral perspective view of the patch portion of the device shown in FIG. 8 and FIG. 9 in a post-deployment configuration (FIG. 10); a lateral perspective view of a strut and clip of the patch portion of the device shown in FIG. 9 (FIG. 11); a cross-sectional view of the strut shown in FIG. 11 (FIG. 12); a close-up, partial, cutaway, perspective view of the second end of the strut shown in FIG. 11 shown in the pre-deployment configuration (FIG. 13); a close-up, partial, cutaway, perspective view of the second end of the strut shown in FIG. 11 shown in the post-deployment configuration (FIG. 14); a lateral perspective view of a stabilization wire used in the device (FIG. 15); a lateral perspective view of the strut and clip of the patch portion of the device shown in FIG. 11 with the stabilization wire shown in FIG. 15 in position within the strut (FIG. 16); a cross-sectional view of the mid section of the strut shown in FIG. 16 (FIG. 17); and a lateral perspective view of a group of stabilization wires, as shown in FIG. 15, joined together centrally by a fitting to a central pull wire (FIG. 18). As can be seen, in this embodiment, the device 200 comprises a patch portion 202 comprising a first side 204, an opposing second side 206, a plurality of struts 208 arranged radially between the first side 204 and the second side 206, and a clip 210 associated with each strut. Except as disclosed in this disclosure, the embodiment of the device 200 is constructed and functions similarly to the embodiment of the device 100 of the present invention.

In one embodiment, the first side 204, the second side 206 or both the first side 204 and the second side 206 comprise material selected from the group consisting of polypropylene, polypropylene mesh, polytetrafluoroethylene (PTFE) graft material and silicone rubber. In a preferred embodiment, such as when the device 200 is being used to obliterate an inguinal hernia sac in a human, the first side 204 comprises polypropylene mesh and the second side 206 comprises polytetrafluoroethylene. The first side 204 and the second side 206 can, however, comprise any suitable material, as will be understood by those with skill in the art with reference to this disclosure.

Each strut 208 comprises a first end 212 and a second end 214. The patch portion 202 has a center and a circumference. The first end 212 of each strut 208 is oriented toward the center of the patch portion 202, and the second end 214 is oriented toward the circumference of the patch portion 202. Each strut 208 has a clip 210 associated with the second end 214 of the strut 208 to attach the patch portion 202 to a surface or structure adjacent to or within the aberrant space or cavity to be obliterated, such as to the peritoneal surface at the entry site into the hernia sac of an inguinal hernia, thereby immobilizing the patch portion 202 of the device 200 in position.

In one embodiment, the device 200 comprises two or more than two struts 208. In another embodiment, the device 200 comprises between 2 and 20 struts 208. In another embodiment, the device 200 comprises between 5 and 8 struts 208.

FIG. 8, FIG. 9 and FIG. 10 show the device 200 in a post-deployment configuration. As can be seen with particular reference to FIG. 10, in a preferred embodiment, when this embodiment is in a post-deployment configuration, the patch portion 202 is convex toward the first side 204 and comprises a generally circular circumference, though other configurations are suitable, as will be understood by those with skill in the art with reference to this disclosure.

In a preferred embodiment, the second side 206 comprises visually discernable markings 216, such as for example colored dots, corresponding to the position of each clip 210.

Referring now to FIG. 11 through FIG. 14, each strut 208 is curved or bent to create the convex shape of the patch portion 202 in the post-deployment configuration. Each strut comprises material suitable for maintaining the desired shape of the patch portion 202. In a preferred embodiment, such as when the device 200 is used to obliterate an aberrant space or cavity within a human, each strut 208 comprises polypropylene or another suitable biocompatible polymer.

As can be seen best in FIG. 12, in one embodiment, each strut 208 comprises two lumens, a first lumen 218 and a second lumen 220. The first lumen 218 is configured to contain a stabilization wire 222. The second lumen 220 is configured to contain an attachment line 224.

Referring now particularly to FIG. 13 and FIG. 14, the clip 210 corresponds to the clip 116 disclosed in connection with the embodiment of the device 100, and comprises a plurality of arm 226 comprising a first end 228 and a second end 230. The first ends 228 of each arms 226 of each clip 210 are joined together, and are also connected to the attachment line 224. In a preferred embodiment, the second end 230 of each arm 226 comprises one or more than one gripping tip, such as, for example, a sharp point or barb. Though shown with only two arms 226, in one embodiment, each clip 210 comprises three or more arms, as will be understood by those with skill in the art with reference to this disclosure. In one embodiment, each clip 210 comprises tantalum, titanium or another suitable metal; however, each clip 210 can comprise any suitable material, as will be understood by those with skill in the art with reference to this disclosure.

As can be seen with particular reference to FIG. 13, in the pre-deployment position, the second ends 230 of each arm 226 are separated from each other and extend maximally outside the second end 214 of the strut 208. As can be seen with particular reference to FIG. 14, after deployment, axial force is applied to the attachment line 224 toward the center of the patch portion 202, thereby translating the joined first ends 212 of the clip 210 axially toward the center of the patch portion 202 within the strut 208, and approximating the second ends 230 of the arms 226 of the clip 210 by crimping the clip 210 at the joined first ends 228. Any surface or structure adjacent to or within the aberrant space or cavity to be obliterated between the second ends 230 of the clip 210 at the time of deployment is then caught between the second ends 230, thereby immobilizing the device 200 in position. Each attachment line 224 is joined at the proximal end to form a collective attachment line allowing simultaneous closing of the second ends 230 of the arms 226 of multiple clips 210.

Referring now to FIG. 15, there is shown a lateral perspective view of a stabilization wire 222 used with the device 200. The stabilization wire 222 assists in positioning and in deploying the device 200. In one embodiment, the stabilization wire 222 comprises a superelastic shape memory material, such as a shaped metal alloy, or comprises a shaped memory polymer, such as a suitable polystyrene material. In a preferred embodiment, the metal alloy is selected from the group consisting of nitinol and Elgiloy®. In another preferred embodiment, the stabilization wire 222 has an outer diameter of between about 0.2 mm and 0.35 mm. In a preferred embodiment, there is provided one stabilization wire 222 for each strut 208. The stabilization wire 222 comprises a proximal end 232 and a distal end 234. As can be seen particularly in FIG. 18, the proximal ends 232 of each stabilization wire 222 are joined to a central pull wire 236 by a small metal fitting 238.

According to another embodiment of the present invention, there is provided a deployment system for deploying the device 200 for the obliteration of an aberrant space or cavity according to the present invention. Referring now to FIG. 19, there is shown a partial, lateral perspective view of a deployment system 240 according to the present invention. As can be seen, the deployment system 240 comprises a device 200 according to the present invention. The deployment system 240 further comprises a deployment catheter 242 comprising a proximal end 244 and a distal end 246. The deployment catheter 242 tapers from its proximal end 244 to its distal end 246, such as for example being 8 F (2.7 mm) at its proximal end 244 and 4 F to 5 F (1.35 mm to 1.67 mm) at its distal end 246. Preferably, the deployment catheter 242 is very stiff. The deployment catheter 242 is configured to advance over a guidewire 248, such as for example a 1 mm guidewire, in monorail fashion, as will be understood by those with skill in the art with reference to this disclosure. The deployment system 240 further comprises one stabilization wire 222 within each of the plurality of struts 208, where the stabilization wires 222 are joined to a central pull wire, such as for example by a small metal fitting 238, within the deployment catheter 242.

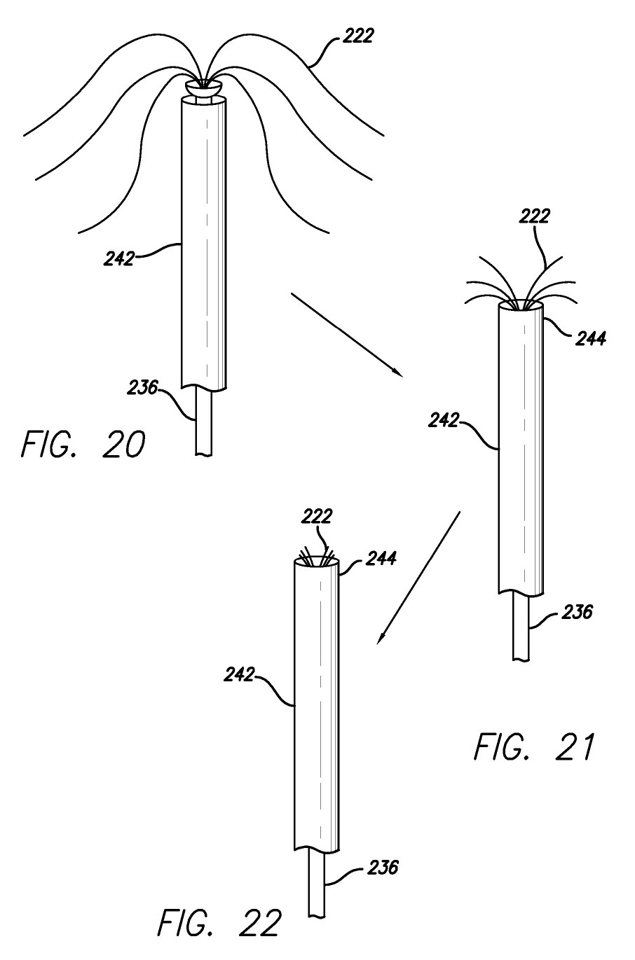

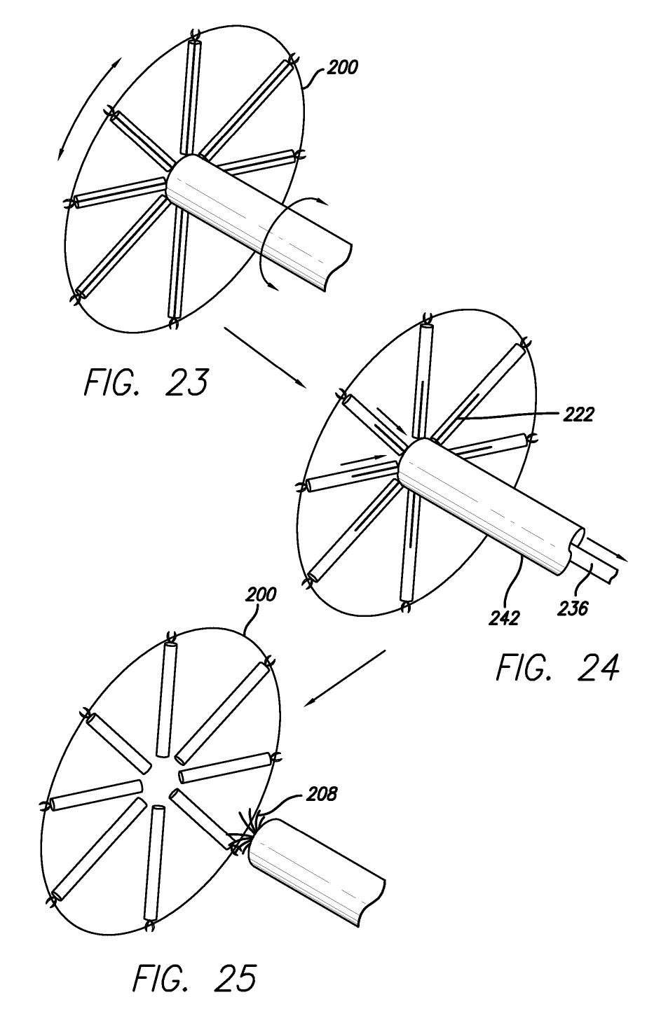

Referring now to FIG. 20, FIG. 21 and FIG. 22, there are shown partial, lateral perspective views of the stabilization wires 222, central pull wire 236 and metal fitting 238 as they are retracted into the proximal end 244 of the deployment catheter 242. The stabilization wires 222 and deployment catheter 242 function to allow precise rotation and positioning of the device 200, to maintain the shape and rigidity of the device 200 while the clips 210 are being crimped to engage the material around the opening of the aberrant space or cavity thereby obliterating the opening, and to allow the struts 208 to detach from the device 200, thereby leaving the device 200 in position after sealing the opening.

Referring now to FIG. 23, FIG. 24 and FIG. 25, there are shown partial, lateral perspective views of the proximal end of the deployment system 240, showing the device 200 being rotated for proper placement (FIG. 23); showing the stabilization wires 222 being withdrawn from the struts 208 into the proximal end 244 of the deployment catheter 242 (FIG. 24); and showing the deployment catheter 242 with the struts 208 inside being separated from the device 200 (FIG. 25). The deployment catheter 242 also transmits the attachment line 224 (not shown in these Figures) for crimping the clips 210.

According to another embodiment of the present invention, there is provided another method for the obliteration of an aberrant space or cavity comprising an open end and a closed end. In a preferred embodiment, the aberrant space or cavity obliterated by the method is within a living organism, such as within a human. In another preferred embodiment, the aberrant space or cavity obliterated by the method is a hernia sac of an inguinal hernia. In one embodiment, the method comprises providing a device according to the present invention, and deploying the device to substantially seal the open end of the aberrant space or cavity. In another embodiment, the method comprises creating an opening in the closed end of the aberrant space or cavity, introducing a device through the opening in the closed end of the aberrant space or cavity, and deploying the device to substantially seal the open end of the aberrant space or cavity. In a preferred embodiment, the device introduced is a device according to the present invention.

By way of example, the method will now be disclosed in greater detail with specific reference to the obliteration of a hernia sac of an inguinal hernia. As will be understood by those with skill in the art with reference to this disclosure, however, equivalent steps can be used to obliterate any aberrant space or cavity suitable for obliteration by the present method, including an aberrant space or cavity other than the hernia sac of an inguinal hernia, other than within a living organism, and other than within a human.

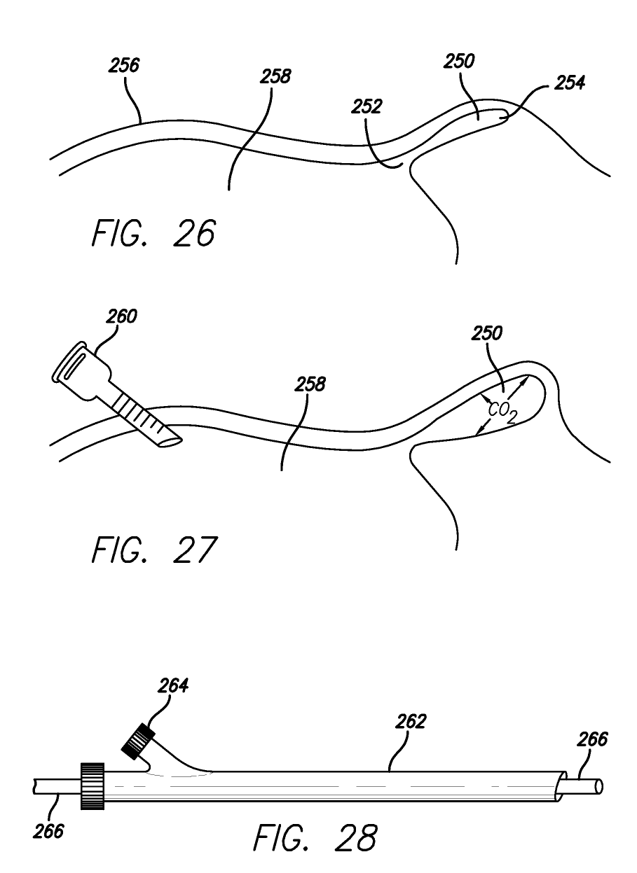

The method comprises, first selecting an aberrant space or cavity that is suitable for obliteration by the method. Referring now to FIG. 26, there is shown a cross-sectional view of an aberrant space or cavity suitable for obliteration by a method for the obliteration of an aberrant space or cavity according to the present invention. As can be seen, the aberrant space or cavity 250 comprises an open end 252 and a closed end 254. In one embodiment, the aberrant space or cavity is within a living organism. In a preferred embodiment, the aberrant space or cavity is within a human. In a particularly preferred embodiment, as shown in FIG. 26, the aberrant space or cavity 250 is a hernia sac of an inguinal hernia within a human.

In one embodiment, selecting an aberrant space or cavity that is suitable for obliteration by the method comprises selecting a patient having a disease or condition that includes the existence of an aberrant space or cavity, such as, for example, the hernia sac of an inguinal hernia, and for which treatment of the disease or condition involves obliteration of the aberrant space or cavity. In this embodiment, selecting the patient comprises diagnosing the existence of an aberrant space or cavity using standard techniques, such as a technique selected from the group consisting of CT scan, herniography, history, MRI and physical examination.

The following steps for the present method are disclosed with respect to obliterating the hernia sac of an inguinal hernia as an example only of one type of aberrant space or cavity suitable for obliteration by the present method. After the patient having the inguinal hernia is selected, anesthesia is induced, and the lower abdomen 256 is prepped and draped in a sterile fashion according to standard techniques. Then, the intraperitoneal cavity 258 as well as the hernia sac 250 is distended with carbon dioxide gas according to standard techniques. Next, as can be seen in FIG. 27, a trocar 260 capable of accommodating a device with an outer diameter of from 28 F to 30 F (9.3 mm to 10.0 mm) is inserted into the intraperitoneal cavity 258 adjacent the inguinal hernia.

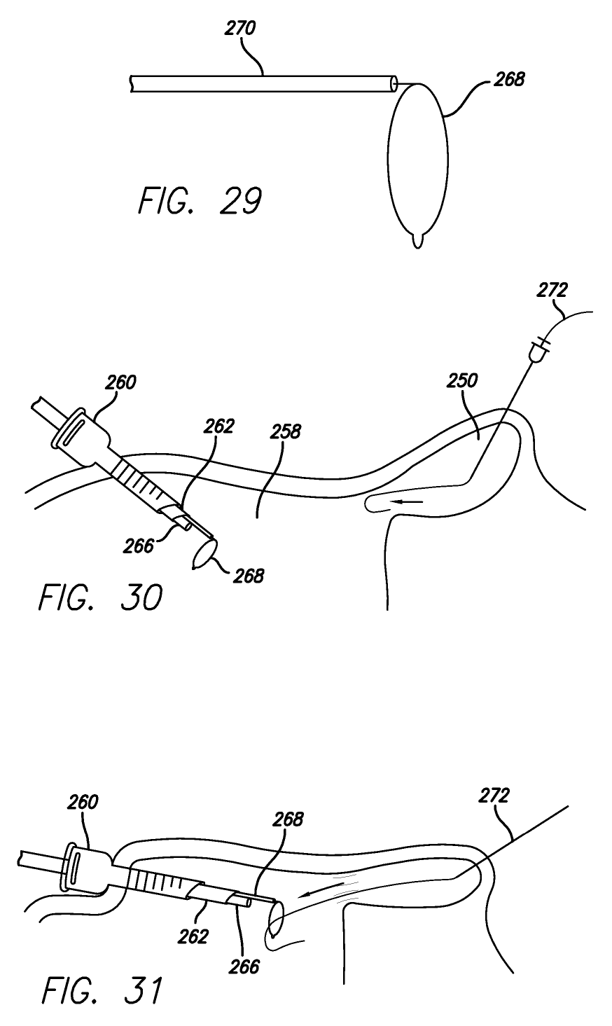

Then, as shown in FIG. 28, an adaptor sheath 262 having two proximal self-sealing valves 264 to prevent leakage of the carbon dioxide gas is provided. Next, the sheath 262 is introduced through the trocar 260 and a 5 mm laparoscope 266 is introduced through one of the valves 264 into the sheath 262 and, thus, into the intraperitoneal cavity 258. Then, an Amplatz™ 90° wire snare 268 (Boston Scientific Corporation, Natick, Mass. US), or equivalent structure, within a 6 F to 8 F (2 mm to 2.7 mm) catheter 270 as shown in FIG. 29 is introduced into the second valve 264 of the trocar 260 into the sheath 262 and, thus, into the intraperitoneal cavity 258 as shown in FIG. 30.

Next, a guidewire 272 of between about 0.45 mm and 0.9 mm and having a proximal end and a distal end is provided, and the guidewire is inserted through the skin over the hernia sac 250 and the proximal end of the guidewire 272 is advanced into the intraperitoneal cavity 258 as shown in FIG. 30. Then, as shown in FIG. 31, under direct vision provided by the laparoscope 266, the wire snare 268 is used to capture the proximal end of the guidewire 272 and to pull the proximal end of the guidewire 272 through the trocar 260 and outside of the body to provide the operator with control over both the proximal end and the distal end of the guidewire 272.

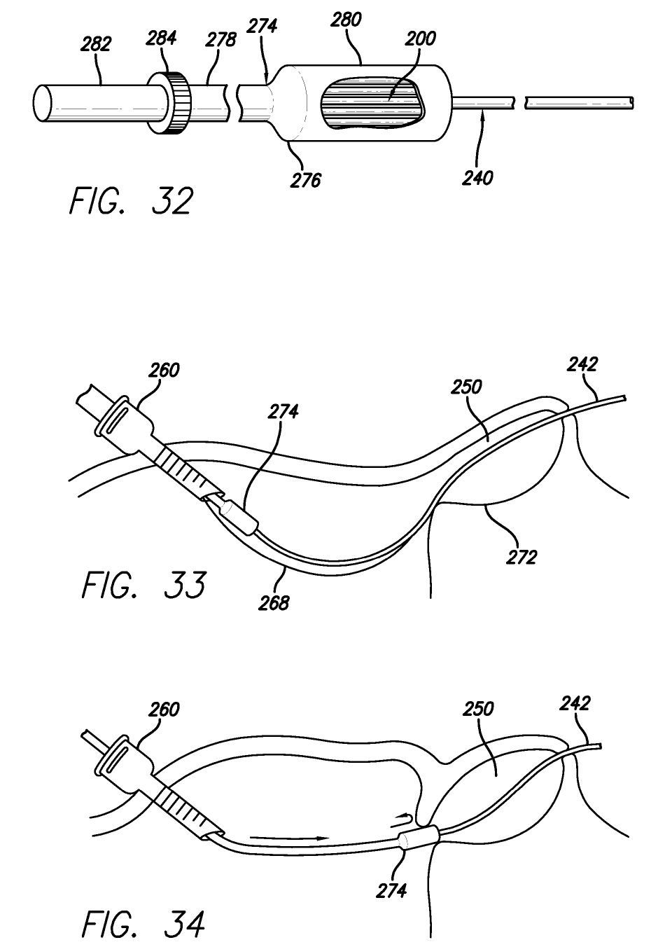

Next, the method comprises providing a delivery device. Referring now to FIG. 32, there is shown a partial, lateral perspective view of a delivery device suitable for use in the present method. As can be seen, the delivery device 274 comprises a capsule 276 having a narrow proximal end 278 and an expanded distal end 280. The delivery device 274 further comprises a pusher 282 at least partly within the proximal end 278 and extending outward from the proximal end 278 as shown. The delivery device 274 further comprises a rotating locking adaptor 284 at the proximal end 278 of the capsule 276 for creating a seal between the pusher 282 and the capsule 276. By way of example only, the proximal end 278 of the capsule 276 is about 12 F to 14 F (4 mm to 4.7 mm), the distal end 280 of the capsule 276 is about 28 F to 30 F (9.3 mm to 10 mm), and the pusher 282 is about 10 F. Further as can be seen in FIG. 32, the delivery device 274 contains a deployment system 240 comprising a device 200 according to the present invention in a pre-deployment configuration within the capsule.

Then, the delivery device 274 containing the deployment system 240 is advanced over the guidewire 272 through the trocar 260 using the delivery catheter 242 in monorail fashion as can be seen in FIG. 33. Advancement of the delivery device 274 continues until the distal end 280 of the capsule 276 contacts the area surrounding the open end 252 (peritoneal surface) of the hernia sac 250 as can be seen in FIG. 34.

Next, the deployment system 240 with the device 200 is released from the delivery device 274 by maintaining the pusher 282 in place and retracting the capsule 276 proximally. The patch portion 202 of the device 200 changes to a post-deployment configuration, and the delivery device 274 is withdrawn from the trocar 260 as can be seen in FIG. 35.

Then, the laparoscope 266 is reinserted into the trocar 260 and, under direct vision provided by the laparoscope 266, the patch portion 202 of the device 200 is rotated and otherwise positioned into its final position, such as for example to prevent the clips 210 from engaging neurovascular structures, using the deployment system 240 as shown in FIGS. 23-25 and in FIG. 36. Next, as can be seen in FIG. 37, the arms 226 of the clips 210 are crimped or otherwise activated, such as for example by retracting the attachment lines 224 (not shown) to engage the area surrounding the open end 252 (peritoneal surface) of the hernia sac 250, thereby closing the open end 252 and effecting the obliteration of the aberrant space or cavity. Then, the deployment catheter 242, the stabilization wires 222, the laparoscope 266 and the trocar 260 are withdrawn, and the entry sites in the lower abdomen 256 are closed using standard techniques, as will be understood by those with skill in the art with reference to this disclosure.

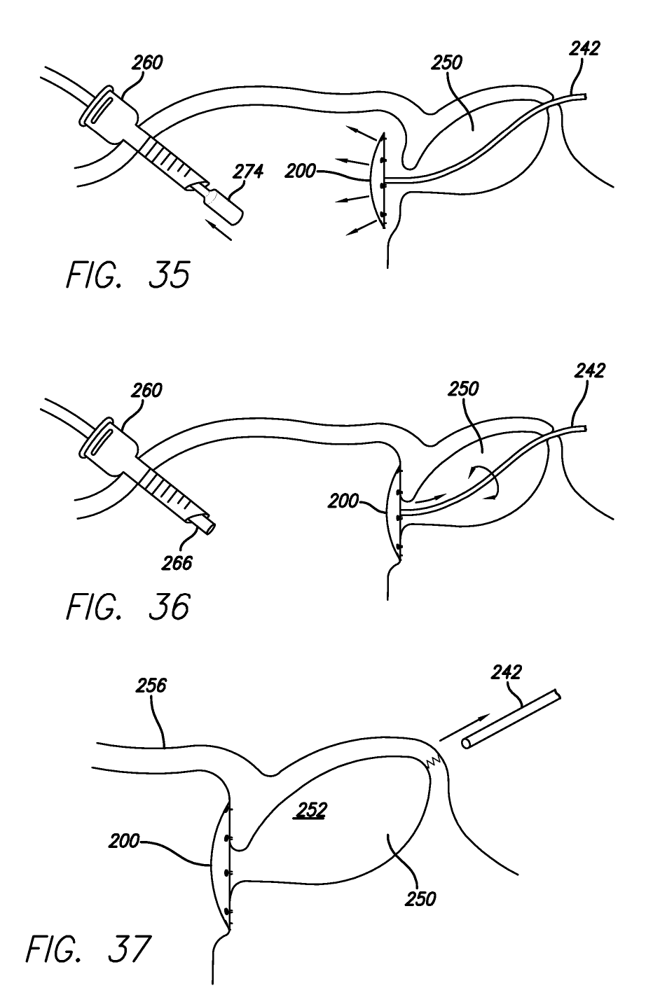

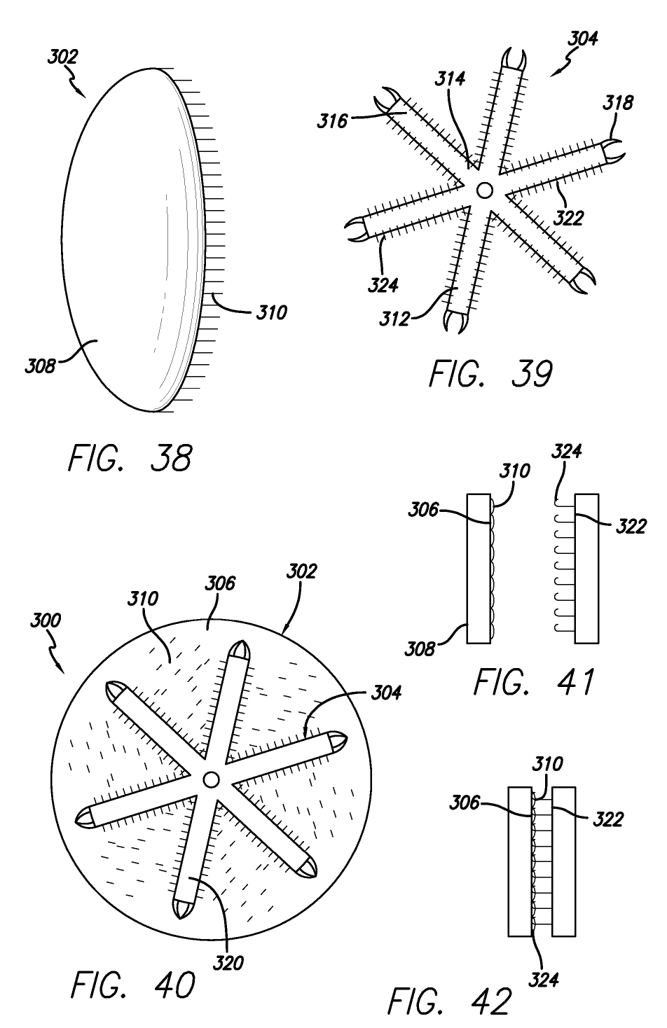

Referring now to FIG. 38 through FIG. 42, there are shown, respectively, a lateral perspective view of the second side of the patch portion of another embodiment of the device for the obliteration of an aberrant space or cavity in a post-deployment configuration (FIG. 38); a top perspective view of the frame portion of the embodiment of the device shown in FIG. 38 in a post-deployment configuration (FIG. 39); a bottom perspective view of the patch portion of the device shown in FIG. 38 and the frame shown in FIG. 39 in a post-deployment configuration, where the patch portion and frame portion are joined together (FIG. 40); a partial lateral perspective view of the opposing surfaces of a hook and loop fastener with the two sides in the unattached configuration (FIG. 41); and a partial lateral perspective view of the opposing surfaces of a hook and loop fastener as shown in FIG. 41 with the two sides in the attached configuration (FIG. 42). As can be seen, in this embodiment, the device 300 comprises a patch portion 302 and a frame portion 304. The patch portion 302 comprises a first side 306, and an opposing second side 308. Except as disclosed in this disclosure, the embodiment of the device 300 is constructed and functions in a manner corresponding to the embodiments of the device 100 and device 200 of the present invention, as will be understood by those with skill in the art with reference to this disclosure.

In one embodiment, the first side 306 and the second side 308 comprise material selected from the group consisting of polypropylene, polytetrafluoroethylene (PTFE) graft material and silicone rubber. In a preferred embodiment, such as when the device 300 is being used to obliterate an inguinal hernia sac in a human, the first side 306 comprises polypropylene and the second side 308 comprises polytetrafluoroethylene. The first side 306 and the second side 308 can, however, comprise any suitable material, as will be understood by those with skill in the art with reference to this disclosure.

In a preferred embodiment, the first side 306 of the device 300 comprises an expansile skeleton, corresponding to the frame 106 of the embodiment of the device 100 disclosed above, or comprises an inflatable structure or comprises an equivalent structure, where the expansile skeleton functions to permit the patch portion 302 of the device 300 to be put into a low profile, pre-deployment, configuration similar to the low profile, pre-deployment, configuration of the embodiments of the device 100 and the device 200, as will be understood by those with skill in the art with reference to this disclosure.

The first side 306 of the patch portion 302 comprises a first surface 310 of two opposing surfaces of a hook and loop fastener, such as for example the hook and loop fastener known under the trademark VELCRO® (Velcro Industries, Curacao, Netherlands Antilles), though any equivalent fastener can be used, as will be understood by those with skill in the art with reference to this disclosure. As shown in FIG. 41 and in FIG. 42, in one embodiment, the first surface 310 is the female surface of the hook and loop fastener; however, in another embodiment, not shown, the first surface 310 is the male surface of the hook and loop fastener.

Referring again to FIG. 39, the device 300 further comprises a frame portion 304. The frame portion 304 comprises a plurality of peripherally radiating members 312 comprising a first end 314 and a second end 316. The first ends 314 of each radiating member 312 are joined together. In a preferred embodiment, as shown in FIG. 39, each second end 112 of the peripherally radiating members 312 comprises a structure, such as a clip 318 or other equivalent structure to attach the radiating member 312, and hence the frame portion 304, to a surface or structure adjacent to or within the aberrant space or cavity to be obliterated, such as to the peritoneal surface at the entry site into the hernia sac of an inguinal hernia, thereby immobilizing the device 300 in position. As will be understood by those with skill in the art with reference to this disclosure, the clip 318 or other equivalent structure corresponds to the clip 116 or the clip 210 as disclosed in this disclosure, as will be understood by those with skill in the art with reference to this disclosure.

Referring again to FIG. 39 and to FIG. 41 and 42, the frame portion 304 further comprises a first side 320 and an opposing second side 322. The second side 322 comprises a second surface 324 of two opposing surfaces of a hook and loop fastener, such as for example the hook and loop fastener known under the trademark VELCRO® (Velcro Industries, Curacao, Netherlands Antilles), though any equivalent fastener can be used, as will be understood by those with skill in the art with reference to this disclosure, where the second surface 324 is configured to mate with the first surface 310 of the first side 306 of the patch portion 302 as disclosed in this disclosure. As shown in FIG. 41 and in FIG. 42, in one embodiment, the second surface 324 is the male surface of the hook and loop fastener; however, in another embodiment, not shown, the second surface 324 is the female surface of the hook and loop fastener.

Referring now to FIG. 40, when contacted together, the first surface 310 on the first side 306 of the patch portion 302 joins to the second surface 324 on the second side 322 of the frame portion 304, thereby attaching the patch portion 302 of the device 300 to the frame portion 304 of the device 300.

According to another embodiment of the present invention, there is provided a method for the obliteration of an aberrant space or cavity comprising an open end and a closed end. In a preferred embodiment, the aberrant space or cavity obliterated by the method is within a living organism, such as within a human. In another preferred embodiment, the aberrant space or cavity obliterated by the method is a hernia sac of an inguinal hernia. In one embodiment, the method comprises providing a device according to the present invention, and deploying the device to substantially seal the open end of the aberrant space or cavity. In another embodiment, the method comprises creating an opening in the closed end of the aberrant space or cavity, introducing a device through the opening in the closed end of the aberrant space or cavity, and deploying the device to substantially seal the open end of the aberrant space or cavity. In a preferred embodiment, the device introduced is a device according to the present invention.

By way of example, the method will now be disclosed in greater detail with specific reference to the obliteration of a hernia sac of an inguinal hernia. As will be understood by those with skill in the art with reference to this disclosure, however, equivalent steps can be used to obliterate any aberrant space or cavity suitable for obliteration by the present method, including an aberrant space or cavity other than the hernia sac of an inguinal hernia, other than within a living organism, and other than within a human.

As will be appreciated by one with skill in the art with reference to this disclosure, the method of delivering and placing the device 300 can include an open procedure, a percutaneous transcatheter procedure, a laparoscopic procedure or a combination of the preceding procedures. Regardless of the procedure used, the method comprises, first selecting an aberrant space or cavity that is suitable for obliteration by the method. The aberrant space or cavity comprises an open end and a closed end. In one embodiment, the aberrant space or cavity is within a living organism. In a preferred embodiment, the aberrant space or cavity is within a human. In a particularly preferred embodiment, the aberrant space or cavity is a hernia sac of an inguinal hernia within a human, though any other suitable aberrant space or cavity can be obliterated.

In one embodiment, selecting an aberrant space or cavity that is suitable for obliteration by the method comprises selecting a patient having a disease or condition that includes the existence of an aberrant space or cavity, such as, for example, the hernia sac of an inguinal hernia, and for which treatment of the disease or condition involves obliteration of the aberrant space or cavity. In this embodiment, selecting the patient comprises diagnosing the existence of an aberrant space or cavity using standard techniques, such as a technique selected from the group consisting of CT scan, herniography, history, MRI and physical examination.

Then, the frame portion 304 of the device 300 is positioned at the open end of the aberrant space or cavity. Next, the frame portion 304 is attached to a surface or structure adjacent to or within the aberrant space or cavity to be obliterated, such as to the peritoneal surface at the entry site into the hernia sac of an inguinal hernia, thereby immobilizing the frame portion 304 in position. Then, the patch portion 302 is introduced near the surface or structure adjacent to or within the aberrant space or cavity, and the patch portion 302 is converted from the pre-deployment configuration to the post-deployment configuration using techniques as will be understood by those with skill in the art with reference to this disclosure. Next, the patch portion 302 is positioned against the frame portion 304, such that the first surface 310 on the first side 306 of the patch portion 302 contacts the second surface 324 on the second side 322 of the frame portion 304, thereby attaching the patch portion 302 of the device 300 to the frame portion 304 of the device 300, and thereby obliterating the cavity.

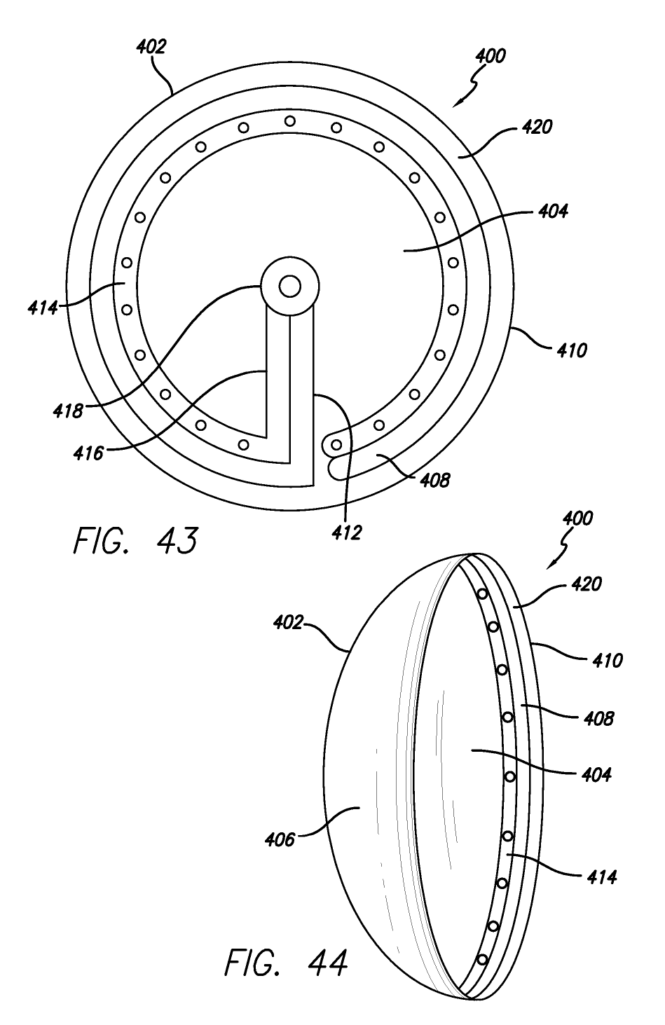

Referring now to FIG. 43 and FIG. 44, there are shown, respectively, a top perspective view of the first side of the patch portion of another embodiment of the device for the obliteration of an aberrant space or cavity in a post-deployment configuration (FIG. 43); and a lateral perspective view of the patch portion of the device shown in FIG. 43 in a post-deployment configuration (FIG. 44). As can be seen, in this embodiment, the device 400 comprises a patch portion 402 comprising a first side 404 and an opposing second side 406, and further comprising an outer edge. Except as disclosed in this disclosure, the embodiment of the device 400 is constructed and functions similarly to the embodiments of the device 100, device 200 and device 300 of the present invention. Though shown as having a generally circular outer circumference, the patch portion 402 can be any shape suitable for its intended use, as will be understood by those with skill in the art with reference to this disclosure. Further, the patch portion 402 can be any size suitable for its intended use, as will be understood by those with skill in the art with reference to this disclosure. In one embodiment, the patch portion 402 has a maximum lateral extent of between 1 cm and 10 cm. In another embodiment, the patch portion 402 has a maximum lateral extent of greater than 5 cm. In another embodiment, the patch portion 402 has a maximum lateral extent of greater than 10 cm.

In one embodiment, the first side 404 and the second side 406 comprise material selected from the group consisting of polypropylene, polytetrafluoroethylene (PTFE) graft material and silicone rubber. In a preferred embodiment, as shown, such as when the device 400 is being used to obliterate an inguinal hernia sac in a human, the first side 404 comprises polypropylene mesh and the second side 406 comprises polytetrafluoroethylene (PTFE). The first side 404 and the second side 406 can, however, comprise any suitable material, as will be understood by those with skill in the art with reference to this disclosure.

The patch portion 402 of the device 400 further comprises an inflation area or inflation channel 408. When inflated, the inflation area or inflation channel 408 gives shape to the patch portion 402 of the device 400 in a post-deployment configuration, thereby corresponding in function to the frame 106 of the device 100, the struts 208 of the device 200, and the peripherally radiating members 312 of the device 300.

As can be seen in FIG. 43, in one embodiment, the inflation area or inflation channel 408 is a circumferential conduit arrayed just central to the outer edge 410 of the patch portion 402 of the device 400. In a preferred embodiment, the inflation area or inflation channel 408 comprises a single, centrally orientated inflation arm 412, as shown in FIG. 43. The inflation area or inflation channel 408 can, however, be any shape or area, or series of multiple shapes or areas suitable for use with the device 400, as will be understood by those with skill in the art with reference to this disclosure.

The patch portion 402 of the device 400 further comprises an adhesive delivery channel 414. When the device is in the post-deployment configuration, adhesive is introduced into the adhesive delivery channel 414, thereby attaching the patch portion 402 of the device 400 to the area surrounding the open end of the aberrant space or cavity and obliterating the aberrant space or cavity. Therefore, the adhesive delivery channel 414 corresponds in function to the clips 116 of the device 100, and the clips 210 of the device 200, and the clips 318 of the device 300 (as well as the hook and loop fastener system of the device 300).

As can be seen in FIG. 43, in a one embodiment, the adhesive delivery channel 414 is a circumferential conduit arrayed just central to the inflation area or inflation channel 408 of the device 400. In a preferred embodiment, the adhesive delivery channel 414 comprises a single, centrally orientated adhesive delivery arm 416, as shown in FIG. 43. The adhesive delivery channel 414 can, however, be any shape or area, or series of multiple shapes or areas suitable for use with the device 400, as will be understood by those with skill in the art with reference to this disclosure. The adhesive delivery channel 414 comprises a series of perforations to allow adhesive to exit from the adhesive delivery channel 414.

The patch portion 402 of the device 400 further comprises one or more than one valve 418 for introducing inflation material into the inflation area or inflation channel 408 and for introducing adhesive into the adhesive delivery channel 414. In a preferred embodiment, the one or more than one valve is a single valve 418, as shown in FIG. 43. In a preferred embodiment, the one or more than one valve 418 is self-sealing and is detachable. The valve 418 can be placed anywhere on the patch portion 402 that is suitable for its intended use. In a preferred embodiment, the inflation area or inflation channel 408 comprises a single, centrally orientated inflation arm 412, and the adhesive delivery channel 414 comprises a single, centrally orientated adhesive delivery arm 416, and the one or more than one valve 418 is positioned in the center of the patch portion 402 in continuity with the centrally orientated inflation arm 412 and the centrally orientated adhesive delivery arm 416, as shown in FIG. 43.

In a preferred embodiment, the patch portion 402 of the device 400 further comprises a peripheral flange 420, oriented peripherally to the inflation area or inflation channel 408. The first side 404 and second side 406 are tightly joined together at the flange 420, and do not separate when the inflation area or inflation channel 408 is inflated in the post-deployment configuration. When present, the flange 420 serves to allow the device 400 to be fixed into position through the flange 420, such as by suturing or clipping the flange 420 to a surface or structure adjacent to or within the aberrant space or cavity to be obliterated, such as to the peritoneal surface at the entry site into the hernia sac of an inguinal hernia, thereby immobilizing the device 400 in position, as will be understood by those with skill in the art with reference to this disclosure.

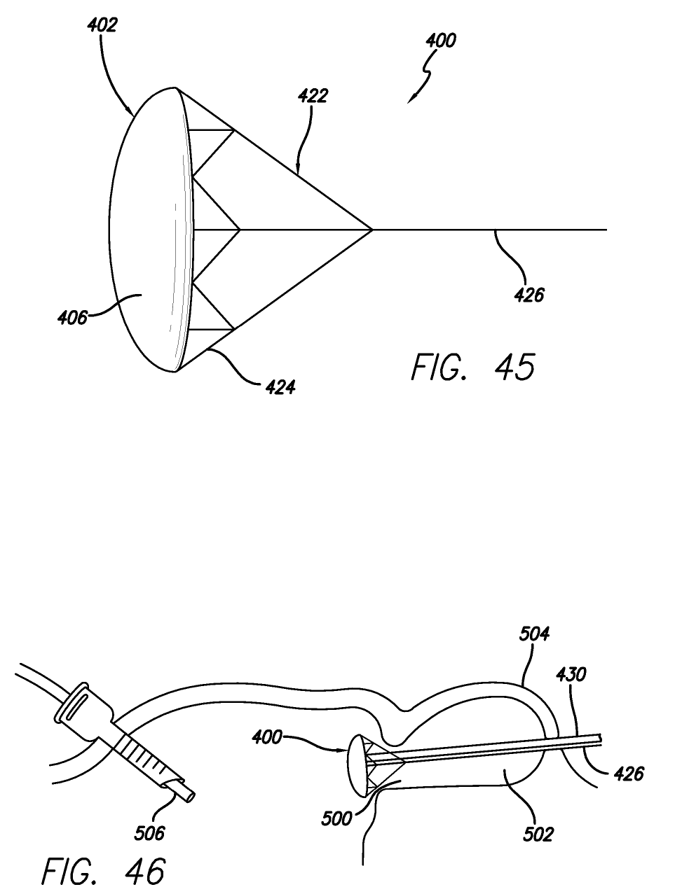

Referring now to FIG. 45, there is shown a lateral perspective view of the patch portion and optional suture harness of the device shown in FIG. 43 in a post-deployment configuration. As can be seen, in one embodiment, the device 400 further comprises a suture harness 422. The suture harness 422 comprises a web of sutures 424 attached to the first side 404 of the patch portion 402 of the device 400. In a preferred embodiment, the device 400 comprises a peripheral flange 420, and the web of sutures 424 attach to the flange 420. In a preferred embodiment, the web of sutures 424 are gathered or joined together to form a central suture or central sutures 426. In a particularly preferred embodiment, the central suture or central sutures 426 is a single central suture 426 as shown in FIG. 45. The suture harness 422 can be used to fix or to assist in fixing into position the device 400 by, for example, securing the central suture 426 into the subcutaneous tissues superficial to an inguinal hernia, or in a corresponding manner when the device 400 is used to obliterate an aberrant space or cavity other than a hernia sac of an inguinal hernia, as will be understood by those with skill in the art with reference to this disclosure.

According to another embodiment of the present invention, there is provided another method for the obliteration of an aberrant space or cavity comprising an open end and a closed end. The method comprises providing a device 400 according to the present invention. Except as disclosed in this disclosure, the method corresponds to the method for the obliteration of an aberrant space or cavity comprising an open end and a closed end as disclosed in connection with the device 200, above.

In summary, the method comprises, first selecting an aberrant space or cavity that is suitable for obliteration by the method. The following steps for the present method are disclosed with respect to obliterating the hernia sac of an inguinal hernia as an example only of one type of aberrant space or cavity suitable for obliteration by the present method. Further, the method steps are disclosed partly with reference to corresponding steps for other embodiments of methods according to the present invention, as disclosed in connection with this disclosure, and as will be understood by those with skill in the art with reference to this disclosure. After the patient having the inguinal hernia is selected, the inguinal hernia sac is distended, and a trocar is inserted into the intraperitoneal cavity as shown in FIG. 27.

Then, a guidewire is introduced through the closed end of the hernia sac and drawn through the trocar to provide the operator with control over both the proximal end and the distal end of the guidewire as shown using the apparatuses and steps in FIGS. 28 through 31. Next, a delivery device corresponding to the delivery device 274 as shown in FIG. 32 is provided containing the device 400 in connection with a deployment system corresponding to the deployment system 240 as shown in FIG. 19. Then, the delivery device containing the deployment system is advanced over the guidewire through the trocar using the delivery catheter part of the deployment system in monorail fashion, and the device 400 is deployed adjacent the open end of the hernia sac as shown in FIG. 33, FIG. 34 and FIG. 35.

Referring now to FIG. 46, there is shown a cross-sectional view of one step in the present method for the obliteration of an aberrant space or cavity according to the present invention. As can be seen, deployment of the device 400 results in the first side 404 of the patch portion 402 and the suture harness 422 of the device 400 facing the open end 500 of the inguinal hernia sac 502. Further, both the distal portion 428 of the deployment catheter 430 and the central suture 426 extend through the hernia sac 502 and out of the skin 504 over the hernia sac 502.

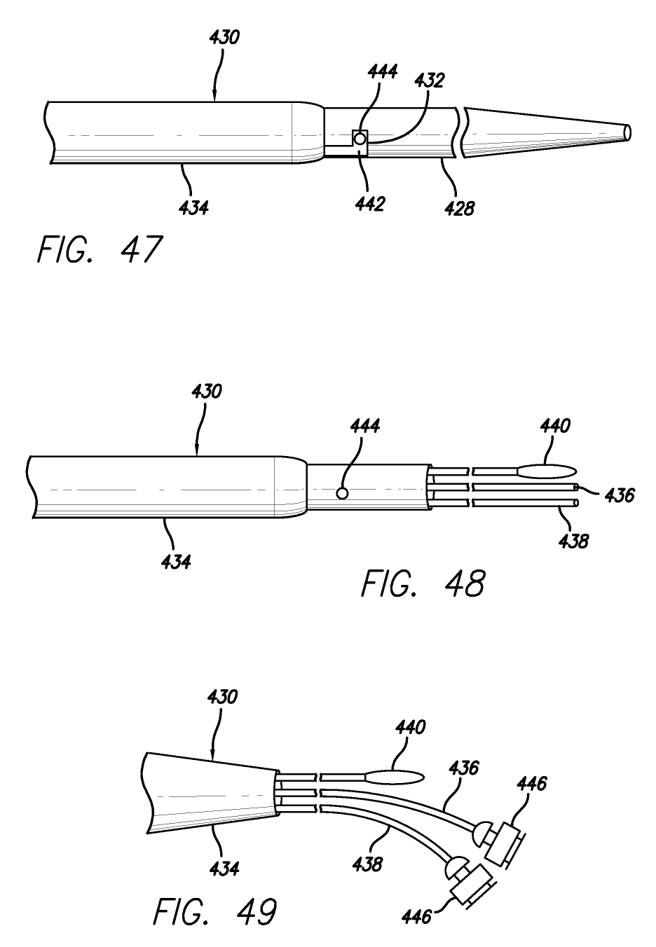

The method now comprises fixing the patch portion 402 of the device 400 to the open end 500 of the hernia sac 502 under guidance of a laparoscope 506. Referring now to FIG. 47, FIG. 48 and FIG. 49, there are shown a partial, close-up lateral perspective view of the distal end of the deployment catheter as shown in FIG. 46 (FIG. 47); a partial, close-up lateral perspective view of the distal end of the deployment catheter as shown in FIG. 47 with the distal portion of the deployment catheter removed (FIG. 48); and a partial, close-up lateral perspective view of the distal end of the deployment catheter as shown in FIG. 48 with luer lock hubs attached to the inflation material delivery conduit and to the adhesive delivery conduit (FIG. 49). As can be seen, in one embodiment, the deployment catheter 430 comprises a locking mechanism 432 that allows an operator to separate the distal portion 428 of the deployment catheter 430 from the proximal portion 434 of the deployment catheter 430, thereby exposing an inflation material delivery conduit 436, an adhesive delivery conduit 438 and a pusher rod 440. In one embodiment, the locking mechanism 432 comprises an indentation 442 on the distal portion 428 of the deployment catheter 430 that mates with a raised protrusion 444 on the proximal portion 434 of the deployment catheter 430 as shown in FIG. 47. In this embodiment, the distal portion 428 of the deployment catheter 430 is separated from the proximal portion 434 by rotating and then axially retracting the distal portion 428 relative to the proximal portion 434. The locking mechanism 432 can, however, be any suitable locking mechanism as will be understood by those with skill in the art with reference to this disclosure. In one embodiment, the deployment catheter comprises a locking mechanism, and the method comprises separating the distal portion of the deployment catheter from the proximal portion, thereby exposing an inflation material delivery conduit, an adhesive delivery conduit and a pusher rod. In this embodiment, the method further comprises attaching luer lock hubs 446 to the inflation material delivery conduit, an adhesive delivery conduit and a pusher rod as seen in FIG. 49.

Next, the method comprises inflating the inflation area or inflation channel of the patch portion of the device to impart structural rigidity to the patch portion. Then, under direct vision provided by the laparoscope, the patch portion of the device is rotated and otherwise positioned into its final position using the deployment catheter in combination with the central suture and pusher rod. Next, adhesive is introduced through the adhesive delivery conduit and the adhesive is allowed to discharge from the series of perforations in the adhesive delivery channel, depositing adhesive between the patch portion of the device and the peritoneal surface adjacent to the open end of the inguinal hernia sac, sealing the patch portion of the device to the peritoneal surface, and thereby, obliterating the inguinal hernia sac. When used in the human body, the adhesive is biocompatible. In one embodiment, the adhesive is n-butyl cyanoacrylate (NBCA) tissue adhesive.

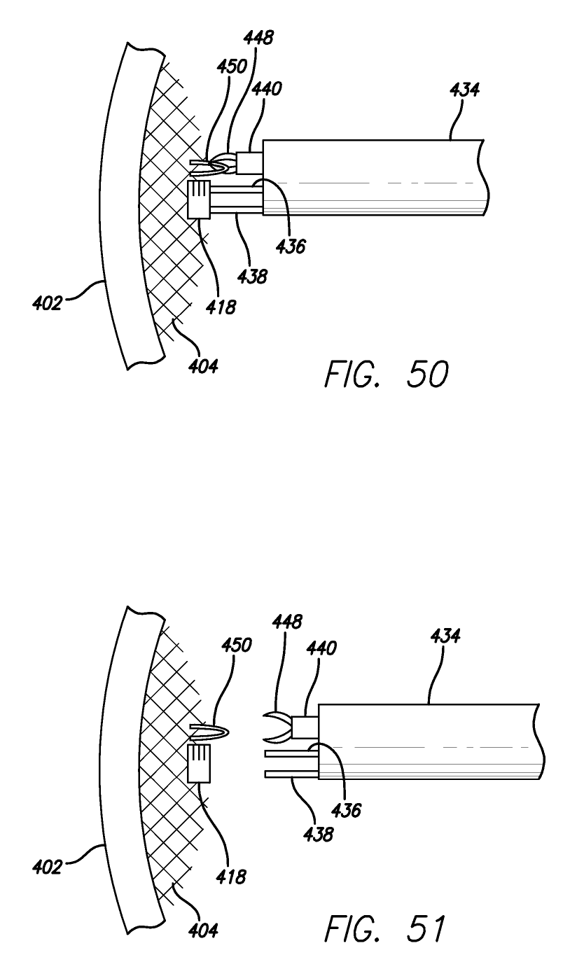

Referring now to FIG. 50 and FIG. 51, there are shown sequential, partial, close-up lateral perspective views of the patch portion of the device in the post-deployment configuration being separated from the proximal portion of the deployment catheter, the inflation material delivery conduit, the adhesive delivery conduit and the pusher rod according to the embodiment of the method shown in FIG. 46. The method then comprises confirming the position of the patch portion of the device using the laparoscope. Next, as can be seen in FIG. 50 and FIG. 51, the patch portion 402 of the device 400 in the post-deployment configuration is separated from the proximal portion 434 of the deployment catheter 430, the inflation material delivery conduit 436, the adhesive delivery conduit 438 and the pusher rod 440. First, the proximal portion 434 is retracted through the skin 504 over the hernia sac 502. Next, the inflation material delivery conduit 436 and the adhesive delivery conduit 438 are detached from the valve or valves 418 using standard techniques, as will be understood by those with skill in the art with reference to this disclosure. Then, the pusher rod 440 is detached from the patch portion 402 of the device 400. Detachment of the pusher rod 440 can be accomplished by exposing a laterally expanding clip 448 attached to loop 450 on the patch portion 402 of the device 400, where exposing the clip 448 occurs when the proximal portion 434 of the deployment catheter 430 is retracted, thereby allowing the clip 448 to expand laterally and detach from the loop 450. Any suitable technique for detaching the pusher rod 440 from the patch portion 402 of the device 400 can, however, be used as will be understood by those with skill in the art with reference to this disclosure. In one embodiment, the device comprises a suture harness with a central suture, and the method further comprises attaching the central suture to the subcutaneous tissue of the skin over the hernia sac.

Finally, the laparoscope and the trocar are withdrawn, and the entry sites in the lower abdomen are closed using standard techniques, as will be understood by those with skill in the art with reference to this disclosure.

Although the present invention has been discussed in considerable detail with reference to certain preferred embodiments, other embodiments are possible. Therefore, the scope of the appended claims should not be limited to the description of preferred embodiments contained in this disclosure. All references cited herein are incorporated by reference to their entirety.

What is claimed is:

1. A device for the obliteration of an aberrant space or cavity comprising:
a) a patch portion comprising a first side and an opposing second side;
b) a plurality of struts arranged radially between the first side and the second side, where each strut comprises a first end and a second end; and
c) a clip associated with the second end of each strut;
where the patch portion has a center and a circumference;
where the first end of each strut is oriented toward the center of the patch portion, and the second end is oriented toward the circumference of the patch portion;
where each clip is configured to attach the patch portion to a surface or structure adjacent to or within the aberrant space or cavity to be obliterated; and
where each strut comprises two lumens, a first lumen configured to contain a stabilization wire, and a second lumen configured to contain an attachment line.

2. The device of claim 1, where the device comprises between 2 and 20 struts.

3. The device of claim 1, where the patch portion is convex toward the first side and comprises a generally circular circumference.

4. The device of claim 1, where the second side comprises visually discernable markings corresponding to the position of each clip.

5. The device of claim 1, where each strut is curved or bent to create the convex shape of the patch portion.

6. The device of claim 1, where each clip comprises a plurality of arms comprising a first end and a second end, where the first ends of each arm of each clip are joined together, and are also connected to an attachment line.

7. The device of claim 6, where the second end of each arm comprises one or more than one gripping tip.

8. The device of claim 6, where each attachment line is joined at the proximal end to form a collective attachment line allowing simultaneous closing of the second ends of the arms of multiple clips.

9. A deployment system for deploying a device according to claim 1, comprising:
a) a device according to claim 1;
b) a deployment catheter comprising a proximal end and a distal end, and configured to advance over a guidewire; and
c) one stabilization wire within each of the plurality of struts, where the stabilization wires are joined to a central pull wire within the deployment catheter.

10. A device for the obliteration of an aberrant space or cavity, the device comprising:
a) a patch portion comprising a first side and an opposing second side; and
b) a frame portion comprising a first side and an opposing second side;
where the first side of the patch portion comprises a first surface of two opposing surfaces of a hook and loop fastener;
where the second side of the frame portion further comprises a second surface of two opposing surfaces of a hook and loop fastener configured to mate with the first surface of the first side of the patch portion;
where the frame portion further comprises a plurality of peripherally radiating members, each of the peripherally radiating members comprising a first end and a second end;
where the first ends of each radiating member are joined together; and
where each of the second ends of the peripherally radiating members comprises a structure to attach the radiating member, and hence the frame portion, to a surface or structure adjacent to or within the aberrant space or cavity to be obliterated.

11. A device for the obliteration of an aberrant space or cavity comprising:
a) a patch portion comprising a first side, an opposing second side, and an outer edge;
b) an inflation area or inflation channel, which when inflated, gives shape to the patch portion in a post-deployment configuration;
c) an adhesive delivery channel comprising a series of perforations to allow adhesive to exit from the adhesive delivery channel; and
d) one or more than one valve for introducing inflation material into the inflation area or inflation channel and for introducing adhesive into the adhesive delivery channel.

12. The device of claim 11, where the inflation area or inflation channel is a circumferential conduit arrayed just central to the outer edge.

13. The device of claim 12, where the inflation area or inflation channel comprises a single, centrally orientated inflation arm.

14. The device of claim 13, where the inflation area or inflation channel comprises a single, centrally orientated inflation arm, and the adhesive delivery channel comprises a single, centrally orientated adhesive delivery arm, and the one or more than one valve is positioned in the center of the patch portion in continuity with the centrally orientated inflation arm and the centrally orientated adhesive delivery arm.

15. The device of claim 14, where the patch portion further comprises a peripheral flange, oriented peripherally to the inflation area or inflation channel to allow the device to be fixed into position through the flange.

16. The device of claim 1, where the device further comprises a suture harness attached to the first side of the patch portion.

17. The device of claim 1, where the patch portion further comprises a peripheral flange, oriented peripherally to the inflation area or inflation channel to allow the device to be fixed into position through the flange;
where the device further comprises a suture harness attached to the first side of the patch portion; and
where the suture harness is attached to the flange.

18. The device of claim 1 or claim 10 or claim 11, where the first side, the second side or both the first side and the second side of the patch portion comprise material selected from the group consisting of polypropylene, polypropylene mesh, polytetrafluoroethylene (PTFE) graft material and silicone rubber.

19. A method for the obliteration of an aberrant space or cavity comprising an open end and a closed end, the method comprising:
a) selecting an aberrant space or cavity that is suitable for obliteration by the method;
b) providing a device according to claim 1 or claim 10 or claim 11; and
c) deploying the device to substantially seal the open end of the aberrant space or cavity.

20. The method of claim 19, where the aberrant space or cavity obliterated by the method is within a living organism.

21. The method of claim 19, where the aberrant space or cavity obliterated by the method is within a human.

22. The method of claim 19, where the aberrant space or cavity obliterated by the method is a hernia sac of an inguinal hernia.

23. The method of claim 19, where the method further comprises creating an opening in the closed end of the aberrant space or cavity and introducing the device through the opening in the closed end of the aberrant space or cavity.

24. The method of claim 19, where the aberrant space or cavity is the hernia sac of an inguinal hernia, where the hernia sac is covered by skin, and where deploying the device comprises:
a) inducing anesthesia;
b) distending the hernia sac with carbon dioxide gas; and
c) inserting a trocar into the intraperitoneal cavity adjacent the inguinal hernia.

25. The method of claim 24, where the method further comprises:
a) providing an adaptor sheath having two proximal self-sealing valves;
b) introducing the sheath through a trocar;
c) introducing a laparoscope through the sheath;
d) introducing a wire snare into the intraperitoneal cavity through the sheath;
e) providing a guidewire having a proximal end and a distal end, and inserting the proximal end of the guidewire through the skin over the hernia sac;
f) advancing the proximal end of the guidewire into the intraperitoneal cavity;
g) capturing the proximal end of the guidewire with the wire snare;
h) pulling the proximal end of the guidewire through the trocar;
i) providing a delivery device comprising a capsule having a proximal end and a distal end and a pusher, and containing a deployment system comprising the device in a pre-deployment configuration within the capsule;
j) advancing the delivery device containing the deployment system over the guidewire through the trocar using the delivery catheter in monorail fashion until the distal end of the capsule contacts the area surrounding the hernia sac;
k) releasing the deployment system with the device from the delivery device by maintaining the pusher in place and retracting the capsule proximally;
l) changing the patch portion of the device to a post-deployment configuration;
m) withdrawing the delivery device from the trocar; and
n) positioning the patch portion of the device, thereby effecting the obliteration of the aberrant space or cavity.

26. A method for the obliteration of an aberrant space or cavity comprising an open end and a closed end, the method comprising:
a) selecting an aberrant space or cavity that is suitable for obliteration by the method;
b) providing a device according to claim 11;
c) deploying the device to substantially seal the open end of the aberrant space or cavity;
d) inflating the inflation area or inflation channel of the patch portion of the device to impart structural rigidity to the patch portion; and
e) introducing adhesive through the adhesive delivery conduit and allowing the adhesive to discharge from the series of perforations in the adhesive delivery channel.
